(19) United States
(12) Patent Application Publication
(10) Pub. No.: US 2011/0129838 A1
(43) Pub. Date: Jun. 2, 2011

(54) USING GENETIC POLYMORPHISMS OF THE BICD1 GENE AS A METHOD FOR DETERMINING A RISK OF DEVELOPING MYOPIA

(75) Inventors: Suh-Hang Hank Juo, Kaohsiung (TW); Chung-Ling Liang, Kaohsiung (TW); Edward Hsi, Kaohsiung (TW)

(73) Assignee: KAOHSIUNG MEDICAL UNIVERSITY, Kaohsiung (TW)

(21) Appl. No.: 13/022,972

(22) Filed: Feb. 8, 2011

Related U.S. Application Data
(63) Continuation-in-part of application No. 12/180,820, filed on Jul. 28, 2008, now Pat. No. 7,906,289.

(51) Int. Cl. C12Q 1/68

(52) U.S. Cl. 435/6.11

(57) ABSTRACT

A method and kit for determining an increased risk of developing myopia in a subject is provided by detecting an SNP in the BICD1 gene. The SNP is selected from a group consisting of rs10844126 (A/C), rs1151029 (A/T), rs2650122 (C/T), rs10771923 (A/G), rs1151009 (T/C), rs2125173 (A/G) and rs161959 (C/G). When the presence of the risk allele associated with myopia is detected at the SNP, the subject is determined in an increased risk of developing myopia.

### CROSS REFERENCE TO RELATED APPLICATIONS

This application is a Continuation-In-Part of pending U.S. patent application Ser. No. 12/180,820, filed Jul. 28, 2008 and entitled “Using Genetic Polymorphisms of The BICD1 Gene as A Method for Diagnosing and Treating Myopia”. The disclosure of the application is incorporated herein by reference.

### BACKGROUND OF THE INVENTION

1. Field of the Invention

The present invention relates to a method for determining an increased risk of developing myopia, and in particular relates to a method for determining an increased risk of developing myopia by determining a genotype of a single nucleotide polymorphism (SNP) in Bicaudal D Homolog 1 (BICD1) gene.

2. Description of the Related Art

Myopia, also called near- or short-sightedness, is a refractive defect of the eye in which collimated light produces image focus in front of the retina when accommodation is relaxed. Those with myopia see nearby objects clearly but distant objects appear blurred. With myopia, the eyeball is too long, or the cornea is too steep, so images are focused in the vitreous inside the eye rather than on the retina at the back of the eye.

Myopia is a common eye condition worldwide. The prevalence of the condition varies widely among populations, genders, and ages (Invest Ophthalmol Vis Sci 1997; 38:334-40; Optom Vis Sci 2001; 78:234-9; J Formos Med Assoc 2001; 100:684-91). In the USA, the prevalence of myopia was estimated to be approximately 25% between ages of 12 to 54 years (Arch Ophthalmol 1983; 101:405-7). In the Baltimore Eye Survey, myopia was less common in blacks (19.4%) compared with whites (28.1%) (Invest Ophthalmol Vis Sci 1997; 38:334-40). High myopia (defined as refractive dioptric power≦−5.0 D in this study) accounted for 27% to 33% of all myopic eyes, corresponding to a prevalence of 1.7% to 2% in the general population in the USA (Arch Ophthalmol 1983; 101:405-7). Taiwan is among the highest risk areas in the world for myopia. Using the definition of less than −6.0 D for high myopia, high myopia is much more common in Asia. The percentage of myopia in Taiwan is 18% among Taiwanese school boys and 24% among Taiwanese school girls (J Formos Med Assoc 2001; 100:684-91). The totals are even higher than the 13.1% reported among young men in Singapore (Optom Vis Sci 2001; 78:234-9). Furthermore, the prevalence of myopia is increasing in Taiwan based on two large nationwide surveys (participant number>10,000) conducted in 1995 and 2000.

High myopia is associated with potential blinding conditions such as retinal detachment, macular degeneration, and glaucoma. It has been estimated that 5.6% of blindness among school children in the USA is attributable to myopia. Substantial resources are required for optical correction of myopia such as spectacles, contact lenses, orthokeratology, photorefractive keratectomy and laser in situ keratomileusis (LASIK). However, these corrections do not prevent the ocular complications mentioned above. Furthermore, complications arising from the use of contact lenses (Curr Opin Ophthalmol 1998; 9:66-71), orthokeratology (Cornea 2003; 22:262-4) and surgical procedures (J Refract Surg 2003; 19:5247-9) also impose additional risks to myopes. In the USA, treatment of myopia costs an estimated $250 million per year (Arch Ophthalmol 1994; 112:1526-30).

While studies have found that several risks were attributed to environmental factors, twin studies have indicated a strong genetic influence on myopia with the estimates of heritability ranging from 58 to 90% (Invest Ophthalmol Vis Sci 2001; 42:1232-6; Genet Epidemiol 1988; 5:171-81; Hum Hered 1991; 41:151-6; Br J Ophthalmol 2001; 85:1470-6). Using family data, it has been reported that a family history was a significant risk factor for high myopia (Invest Ophthalmol Vis Sci 2004; 45:3446-52). Several studies also demonstrated a similar finding (Optom Vis Sci 1996; 73:279-82; JAMA 1994; 271:1323-7; Invest Ophthalmol Vis Sci 2002; 43:3633-40; Optom Vis Sci 1999; 76:387-92; Invest Ophthalmol Vis Sci 2004; 45:2873-8). However, while few papers reported identifying susceptible myopia genes, none of the studies has been replicated, thus making the identification highly questionable.

### BRIEF SUMMARY OF THE INVENTION

The present invention provides a method for determining an increased risk of developing myopia in a subject, which comprises detecting in a sample from the subject at least one of the SNPs in the BICD1 gene. The SNP is selected from a group consisting of rs10844126(A/C), rs1151029(A/T), rs2650122(C/T), rs10771923 (A/G), rs1151009 (T/C), rs2125173 (A/G) and rs161959 (C/G). When the presence of a risk allele is detected at the SNPs, it indicates that the subject has an increased risk of developing myopia. The risk allele comprises the C allele in the SNP rs10844126 (A/C), the T allele in the SNP rs1151029 (A/T), the C allele in the SNP rs2650122 (C/T), the G allele in the SNP rs10771923 (A/G), the C allele in the SNP rs1151009 (T/C), the A allele in the SNP rs2125173 (A/G) or the C allele in the SNP rs161959 (C/G).

The present invention also provides a kit for determining an increased risk of developing myopia in a subject, which comprises a probe or primer that distinguishes the allele of the SNP in the BICD1 gene.

A detailed description is given in the following embodiments.

### DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

In one aspect, the present invention provides a method for determining an increased risk of developing myopia in a subject, which comprises detecting in a sample from the subject at least one of the SNPs in the BICD1 gene. BICD1, Bicaudal D Homolog 1, also known as BICD, is located at chromosome 12 p.11.2-p11.1. The gene is one of two human homologs of Drosophila bicaudal-D and can be queried by an Entrez Gene ID 636 at the website of NCBI (http://www.ncbi.nlm.nih.gov/). BICD1 is conserved in chimpanzees, dogs, cats, mice, rats, chickens, zebra fishes and fruit flies. It has been reported that BICD1 is associated with pancreatic cancer in the Japanese population (Low S K, et al., Genome-wide association study of pancreatic cancer in Japanese population, PLoS One, 2010 Jul. 29.), telomere length homeostasis in humans (Mangino M. et al., BICD1 plays a similar role in telemere length homeostasis in humans, Hum Mol Genet (16), p. 2518-23, 2008) and emphysema (Kong X, et al., Genome-wide Association Study Identifies BICD1 as a susceptibility Gene for emphysema, Am J Respir Crit Care Med, 2010 Aug. 27.). However, there is no prior art disclosing the association between the BICD1 gene and myopia.

According to the invention, the SNP in the BICD1 gene is selected from a group consisting of rs10844126 (A/C), rs1151029 (A/T), rs2650122 (C/T), rs10771923 (A/G), rs1151009 (T/C), rs2125173 (A/G) and rs161959 (C/G). The SNP, single nucleotide polymorphism, refers to a site of one nucleotide that varies among a general population leading to the formation of polymorphism. The rs number represents an internationally known coding system to identify each SNP in the dbSNP of the NCBI's Entrez system. The original database with additional information of the SNP rs number is available at the website of http://www.ncbi.nlm.nih.gov/sites/entrez?db=snp. The rs number can also be queried in the Entrez database such as pubmed (http://www.ncbi.nlm.nih.gov/pubmed/) and GenBank(http://www.ncbi.nlm.nih.gov/).

Following, describes the SNP detected with the chromosomal location (based on genome build GRCh37 using dbSNP build 132. The website of genome build GRCh37 is http://www.ncbi.nlm.nih.gov/projects/genome/assembly/grc/human/data; the website for dbSNP build 132 is http://www.ncbi.nlm.nih.gov/sites/entrez?db=snp) and alleles thereof, by the method of the invention. The SNP rs10844126 is located in the promoter of the BICD1. Its chromosomal position is at 32,256,905 where the nucleotide can be either adenine (A) or cytosine (C). If adenine is present in SNP rs10844126, it is called the A allele is present in this polymorphism. If cytosine is present in SNP rs10844126, it is called the C allele is present in this polymorphism. Since each individual has one pair of the same chromosome, each individual has the SNP rs10844126 genotype either AA, AC or CC. The SNP rs1151029 is located in the first intron of the BICD1 and at the chromosomal position of 32,339,083 where the nucleotide is A or T. The SNP rs2650122 is located in the first intron of the BICD1 and at the chromosomal position of 32,339,891 where the nucleotide is C or T. The SNP rs10771923 is located in the first intron of the BICD1 and at the chromosomal position of 32,320,912 where the nucleotide is A or G. The SNP rs1151009 is located in the first intron of the BICD1 and at the chromosomal position of 32,302,164 where the nucleotide is T or C. The SNP rs2125173 is located in the first intron of the BICD1 and at the chromosomal position of 32,307,918 where the nucleotide is A or G. The SNP rs161959 is located in the first intron of the BICD1 and at the chromosomal position of 32,363,208 where the nucleotide is C or G. Although the information of the SNPs has been recorded in the NCBI database, the clinical association of the SNPs is still unknown, especially for the associations between the SNPs and myopia.

According to the method of the invention, the risk allele associated with myopia includes the C in the SNP rs10844126 (A/C), the T in the SNP rs1151029 (A/T), the C in the SNP rs2650122 (C/T), the Gin the SNP rs10771923 (A/G), the C in the SNP rs1151009 (T/C), the A in the SNP rs2125173 (A/G) and the C in the SNP rs161959 (C/G). The SNPs in the BICD1 gene can be detected individually or combined according to the invention. For higher sensitivity and precision, detection for a combination of SNPs in the BICD1 gene is preferable, but it is not limited thereto.

For determining an increased risk of developing myopia according to the method of the present invention, primer extension (PinPoint assay, Massextend™, SPC-SBE, or GOOD assay), hybridization (TaqMan assay, bead array, or SNP chip), ligation (combinatorial fluorescence energy transfer (CFET) tags), and enzymatric cleavage (RFLP, Invader® assay), PCR-SSCP (single-strand conformation polymorphism), MRD (mismatch repair detection), BeadArray™ , or SNPlex™ can be used. Firstly, a biological sample containing nucleic acid (DNA) is collected from a subject. The subject may be a fruit fly, zebra fish, mouse, rat, chicken and mammal, such as a dog, cow, chimpanzee and human, but it is not limited thereto. A mammal is preferable, while a human is more preferable. The biological sample is isolated or collected from the subject, such as a blood sample, an amniotic fluid, a cerebrospinal fluid, a tissue sample from skin, muscle, buccal, conjunctival mucosa, placenta, gastrointestinal tract or other organs. The DNA sample is then examined to determine whether a polymorphism of the BICD1 gene is present and to determine the presence of the associated genotype in the BICD1 gene. In one embodiment, the presence of the polymorphism in the BICD1 gene can be indicated by TaqMan assay. Briefly, the PCR primers and TaqMan MGB probes are designed with Primer Express version 2.0. Reactions can be performed in 96-well microplates with GeneAmp 9700 thermal cyclers. Fluorescence can be measured with an ABI Prism 7500 sequence detection system and analyzed with the ABI Prism 7500 SDS software version 1.0. In another embodiment, the presence of SNP can be determined by genotyping as described in Mutat Res 2005; 573:70-82. Genotyping can be performed by the Illumina BeadArray technology (Sentrix® Array Matrix) [Shen, 2005 #135]. DNA is annealed to allelic-specific oligonucleotides and amplified by polymerase chain reaction (PCR). Array-based hybridization takes place and genotyping are achieved by Cy-3 and Cy-5 labeled primers. Alternately, a commercial gene chip also can be used to determine presence of SNP in BICD1 gene. The Affymetrix GeneChip® Human Mapping 500K Array Set includes two arrays, each capable of genotyping on average 250,000 SNPs (approximately 262,000 for Nsp arrays and 238,000 for Sty arrays). Genomic DNA is hybridized in accordance with the manufacturer's standard recommendations. Genotypes are determined using BRLMM clustering algorithm.

In addition, if the polymorphism results in the creation or elimination of a restriction site, a restriction digestion can be used to determine the polymorphism in the BICD1 gene. Firstly, the PCR can be used to amplify the BICD1 gene in the biological sample of genomic DNA from the subject. Next, an RFLP analysis is performed. The digestion pattern of the relevant DNA fragment indicates the presence or absence of a particular allele (or genotype) in the BICD1 gene, and therefore indicates an increased or decreased risk of developing myopia. In another embodiment, a sequence analysis can be used to determine the polymorphism in the BICD1 gene. PCR or other appropriate methods can be used to amplify the gene or nucleic acid, and/or its flanking sequences, if desired. The sequence of a BICD1 nucleic acid or a fragment of the nucleic acid, the BICD1 genomic DNA, or fragment of the BICD1 genomic DNA is determined, using standard methods. The presence of the associated alleles in the SNPs indicates that the subject has a susceptibility to myopia and/or myopia related complications.

There are many methods for determining polymorphism in the BICD1 gene. One of ordinary skill in the art will select the appropriate method and protocol to use. These and many other methods will be readily apparent to those of ordinary skill in the art, and are considered as equivalents within the scope of the present invention.

In another aspect, the present invention provides a kit for determining an increased risk of developing myopia in a subject, which comprises a probe or primer that distinguishes the allele of the SNP in the BICD1 gene in a sample from a subject, wherein the allele of the SNP is selected from a group consisting of: A or C in the SNP rs10844126 (A/C), A or T in the SNP rs1151029 (A/T), C or T in the SNP rs2650122 (C/T), A or G in the SNP rs10771923 (A/G), T or C in the SNP rs1151009 (T/C), G or A in the SNP rs2125173 (A/G) and G or C in the SNP rs161959 (C/G).

The term “primer” refers to an oligonucleotide complementary to a nucleic acid strand for initiating the nucleic acid synthesis in the presence of four nucleoside triphosphates, a polymerase and buffer in a hybridization condition. The probe refers to an oligonucleotide that selectively hybridizes to a target nucleic acid under a suitable condition. The probe or primer is not specifically limited in the invention as long as it is capable of distinguishing between the two alleles in one of the SNPs. For easy detection, the probe or primer can be further labeled by fluorescence or detectable materials, such as radioactive materials, chemiluminescence, biotin or the like. The kit can further comprise a polymerase, deoxynucleotides, a restriction enzyme, a buffer or the like for detection. An electronic hardware components, such as arrays (DNA chips), microfluidic systems (“lab-on-a-chip” systems), etc. can also be used for the detection. The kit may further contain means for determining the amount of a target nucleic acid, and means for comparing the amount with a standard, and can comprise instructions for using the kit to detect the SNP-containing nucleic acid molecule of interest.

### EXAMPLES

### Example 1

### Genome Scan in the Initial Step

A total of about 4000 subjects were recruited for the myopia study. The participants were recruited from (1) young men in military conscripts, (2) university students, (3) hospital personnel, (4) patients from ophthalmology clinics and (5) general population. Individuals with spherical refraction≦−6.0 D in one eye and ≦−4.0 D in the other eye were classified as high myopia. A subject was defined as a control if the worse eye had a spherical refraction ≧−1.5 D. All subjects were between the ages of 17˜45 years old. All the participants were of Chinese descent. All participants gave informed consents. The study was approved by the Institutional Review Board at the Kaohsiung Medical University, Kaohsiung, Taiwan.

In the initial step, Affymetrix GeneChip® Human Mapping 500K Array Set was used. It comprised two arrays, each capable of genotyping on average 250,000 SNPs (approximately 262,000 for Nsp arrays and 238,000 for Sty arrays). To ensure the quality of DNA sample, all DNA were required to have the OD 260/280 between 1.7 and 2.0, and 260/230>1.0. About 1.5 μg (30 μl of 50 ng/μl) genomic DNA was required for genotyping. The images were analyzed using BRLMM algorithm to obtain the genotyping data. To remove an SNP or a sample which might have genotyping problems, the following criteria was used: per sample call rate of at least 90%, per SNP call rate of at least 90%, an SNP with the minor allele frequency of at least 1%, and genotypes in Hardy-Weinberg equilibrium (p>0.001). Taken together, 380619 SNPs were considered for further analysis.

To test for allelic and genotypic association between each SNP and the high myopia status, PLINK program (Am J Hum Genet 2007; 81:559-75) was used to calculate genotype and allele frequencies, and to perform χ²test. To investigate the genotypic association under different inheritance models, genotype data were further encoded into dominant, recessive, and additive modes. In addition, the trend test was also performed. The genetic effect along with covariates (sex and age) was included in the logistic regression. A linear regression model was applied to test for the association between each SNP and the refraction errors. Since the refraction errors greater than −3 D were common in the Taiwanese population, the subjects as normal/mild myopia (≧−3 D) and high myopia (≦−6 D) was also dichotomized, and the discrete phenotype was tested in the logistic regression model. For logistic regression analysis, subjects with refraction between −3 and −6 D were not included in the analysis. Hap-clustering was employed to perform haplotype analysis.

For the Affymetrix 500K SNP chips, the average call rate was 98.7 (ranging from 97.4 to 99.5). The initial analysis indicated that the best 10 SNPs had the highest p value=0.0002. The 10 SNPs are on chromosome 1, 2, 4, 6, 12, 16, 17 and 21, and none of them are closely located.

### Example 2

### Genome Scan in the Second Step

In the second step, firstly the 10 best SNPs as the centers was used, and a genomic region of 200 kb surrounding each of the 10 best SNPs as our candidate region was selected. A total of 384 tagging SNPs were selected for follow-up fine mapping in independent 1536 subjects whose refraction errors were either <−6 D or >−1.5 D. Genotyping was performed by the Illumina BeadArray technology (Sentrix® Array Matrix) (Mutat Res 2005; 573:70-82). DNA was annealed to allelic-specific oligonucleotides and amplified by polymerase chain reaction. Array-based hybridization took place and genotyping were achieved by Cy-3 and Cy-5 labeled primers. Thirty replicates of each SNP were done to ensure the highest quality of genotype calling.

### Example 3

### Genome Scan in the Third Step

In the third step, the most promising SNPs based on the stage II result were genotyped by using the TaqMan technology (Applied Biosystems [ABI], Foster City, USA). Briefly, PCR primers and TaqMan MGB probes were designed with Primer Express version 2.0. Reactions were performed in 96-well microplates with GeneAmp 9700 thermal cyclers. Fluorescence was measured with an ABI Prism 7500 sequence detection system and analyzed with the ABI Prism 7500 SDS software version 1.0. The subjects with refraction errors between −6 D and −1.5 D were used in the stage III study.

In the third step, the BICD1 gene was focused and the genetic effect was analyzed using different inheritance models for the seven SNPs in a larger dataset including, SNP rs10844126 in 2323 subjects, SNP rs1151029 in 4131 subjects, SNP rs2650122 in 1950 subjects, SNP rs10771923 in 3640 subjects, SNP rs1151009 in 3260 subjects, SNP rs2125173 in 3260 subjects and SNP rs161959 in 3265 subjects. The allele frequencies of the seven SNPs are listed in Table 1.

Table 2 shows the dichotomized phenotype (≧−3 D as control and ≦−6 D as case) and the genotype of the SNPs with odds ratios (ORs) and p-values from logistic regression analysis. P-values of <0.05 are considered statistically significant. When the phenotype was treated as a continuous trait (that is the refractive error), the seven SNPs showed statistically significant results as illustrated in Table 3.

TABLE 1
Allele frequency and the number of genotyped individuals
for the BICD1 gene

| | Refraction | | −3D to | | |
|---|---|---|---|---|---|
| | errors | ≧−3 D | −6 D | ≦−6 D | Total |
| rs10844126 | sample | 1111 | 167 | 1045 | 2323 |
| | size | | | | |
| | MAF | 47.5% | 51.5% | 52.2% | |
| rs1151029 | sample | 1651 | 1264 | 1216 | 4131 |
| | size | | | | |
| | MAF | 21.3% | 22.9% | 24.8% | |
| rs2650122 | sample | 847 | 140 | 963 | 1950 |
| | size | | | | |
| | MAF | 35.8% | 31.8% | 33.1% | |
| rs10771923 | sample | 1475 | 1062 | 1103 | 3640 |
| | size | | | | |
| | MAF | 37.7% | 40.0% | 42.5% | |
| rs1151009 | sample | 1330 | 1010 | 920 | 3260 |
| | size | | | | |
| | MAF | 20.6% | 22.4% | 23.9% | |
| rs2125173 | sample | 1320 | 1009 | 931 | 3260 |
| | size | | | | |
| | MAF | 29.2% | 27.9% | 25.1% | |
| rs161959 | sample | 1314 | 1019 | 932 | 3265 |
| | size | | | | |
| | MAF | 39.2% | 36.8% | 36.7% | |

MAF: minor allele frequency

TABLE 2
Results of the dichotomized trait (normal/mild vs. high myopia) from logistic regression.

| | Genotype | | | | OR (p-value) | | |
|---|---|---|---|---|---|---|---|
| | N (%) | N (%) | N (%) | Total | Additive | Dominant | Recessive |
| rs10844126 | AA | AC | CC | | | | |
| ≧−3 D | 309 | 549 | 253 | 1111 | 1.21 | 1.27 | 1.29 |
| | (27.8) | (49.4) | (22.7) | | (0.0017) | (0.01326) | (0.0089) |
| ≦−6 D | 242 | 514 | 289 | 1045 | | | |
| | (23.1) | (49.1) | (27.6) | | | | |
| rs1151029 | AA | TA | TT | | | | |
| ≧−3 D | 1034 | 532 | 85 | 1651 | 1.21 | 1.29 | 1.15 |
| | (62.6) | (32.2) | (5.1) | | (0.0018) | (0.0008) | (0.3688) |
| ≦−6 D | 686 | 458 | 72 | 1216 | | | |
| | (56.4) | (37.6) | (5.9) | | | | |
| rs2650122 | CC | TC | TT | | | | |
| ≧−3 D | 341 | 405 | 101 | 847 | 0.88 | 0.80 | 0.99 |
| | (40.2) | (47.8) | (11.9) | | (0.0871) | (0.0224) | (0.9547) |
| ≦−6 D | 439 | 410 | 114 | 963 | | | |
| | (45.5) | (42.5) | (11.8) | | | | |
| rs10771923 | AA | GA | GG | | | | |
| ≧−3 D | 569 | 699 | 207 | 1475 | 1.21 | 1.29 | 1.30 |
| | (38.5) | (47.3) | (14.0) | | (0.0005) | (0.0019) | (0.0137) |
| ≦−6 D | 360 | 549 | 194 | 1103 | | | |
| | (32.6) | (49.7) | (17.5) | | | | |
| rs1151009 | TT | TC | CC | | | | |
| ≧−3 D | 839 | 433 | 58 | 1330 | 1.20 | 1.24 | 1.31 |
| | (63.0) | (32.5) | (4.3) | | (0.0103) | (0.0139) | (0.1625) |
| ≦−6 D | 533 | 335 | 52 | 920 | | | |
| | (57.9) | (36.4) | (5.6) | | | | |
| rs2125173 | AA | GA | GG | | | | |
| ≧−3 D | 656 | 557 | 107 | 1320 | 0.81 | 0.76 | 0.79 |
| | (49.6) | (42.1) | (8.1) | | (0.0022) | (0.0017) | (0.1670) |
| ≦−6 D | 525 | 345 | 61 | 931 | | | |
| | (56.3) | (37.0) | (6.6) | | | | |
| rs161959 | CC | GC | GG | | | | |
| ≧−3 D | 476 | 647 | 191 | 1314 | 0.90 | 0.84 | 0.93 |
| | (36.2) | (49.2) | (14.5) | | (0.1018) | (0.0535) | (0.5916) |
| ≦−6 D | 375 | 429 | 128 | 932 | | | |
| | (40.2) | (46.0) | (13.7) | | | | |

“Additive” means the minor allele of a SNP has an additive effect.
“Dominant” and “recessive” mean the minor allele has the dominant and recessive effect, respectively.

TABLE 3
Results based on the refractive errors in the linear regression model.

| | Additive | | Dominant | | Recessive | |
|---|---|---|---|---|---|---|
| | Allele | | Allele | | Allele | |
| | effect | p-value | effect | p-value | effect | p-value |
| rs10844126 | −0.322 | 0.009 | −0.416 | 0.040 | −0.444 | 0.029 |
| rs1151029 | −0.224 | 0.008 | −0.287 | 0.006 | −0.227 | 0.298 |
| rs2650122 | 0.279 | 0.038 | 0.470 | 0.010 | 0.107 | 0.700 |
| rs10771923 | −0.230 | 0.003 | −0.306 | 0.007 | −0.298 | 0.043 |
| rs1151009 | −0.219 | 0.019 | −0.240 | 0.034 | −0.400 | 0.110 |
| rs2125173 | 0.253 | 0.004 | 0.344 | 0.002 | 0.223 | 0.277 |
| rs161959 | 0.168 | 0.036 | 0.264 | 0.020 | 0.136 | 0.384 |

“Allele effect” means the increase of refractive error (diopter, D) when the presence of one risk allele. For example, the presence of one risk allele C of SNP rs10844126 will increase diopter by −0.322 (D) and the presence of two C alleles (that is this individual's genotype is CC) will increase diopter by −0.644 when the C allele has an additive effect.

According to Tables 2 and 3, the risk allele in each SNP was significantly associated with an increased risk for myopia (all p-values<0.05). For example, the CC genotype of SNP rs10844126 is more common in the high myopic patients and thus an individual carrying the C allele has a higher risk to develop myopia. Similarly, an individual carrying the T of SNP rs1151029, the C allele in the SNP rs2650122, the G allele of SNP rs10771923, the C allele of SNP rs1151009, the A allele of SNP rs2125173, and the C allele of SNP rs161959. The data showed that a strong association exists between these risk alleles and the risk of developing myopia, and, therefore, the detection for the presence of risk allele in the SNP was sufficient to determine the risk of developing myopia in a subject.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for determining an increased risk of developing myopia in a subject, comprising:
detecting in a sample from the subject at least one of SNPs in the BICD1 gene, wherein the SNP is selected from a group consisting of rs10844126(A/C), rs1151029(A/T), rs2650122(C/T), rs10771923 (A/G), rs1151009 (T/C), rs2125173 (A/G) and rs161959 (C/G); and
determining the risk of developing myopia in the subject, wherein the presence of a risk allele is detected at the SNPs and indicates that the subject has an increased risk of developing myopia, wherein the risk allele comprises:
the C allele in the SNP rs10844126(A/C),
the T allele in the SNP rs1151029(A/T),
the C allele in the SNP rs2650122(C/T),
the G allele in the SNP rs10771923 (A/G),
the C allele in the SNP rs1151009 (T/C),
the A allele in the SNP rs2125173 (A/G), or
the C allele in the SNP rs161959 (C/G), and
wherein the myopia comprises a spherical refraction ≦−6 D.

2. The method as claimed in claim 1, wherein the sample comprises blood, an amniotic fluid, cerebrospinal fluid, tissue from skin, muscle, buccal or conjunctival mucosa, placenta, or gastrointestinal tract.

3. The method as claimed in claim 1, wherein the subject comprises mammals.

4. The method as claimed in claim 1, wherein the subject is a human.

5. A kit for determining an increased risk of developing myopia in a subject, comprising a probe or primer that distinguishes an allele of a SNP in the BICD1 gene in a sample from the subject, wherein the allele of the SNP is selected from a group consisting of:
A or C allele in the SNP rs10844126,
A or T allele in the SNP rs1151029,
C or T allele in the SNP rs2650122,
A or G allele in the SNP rs10771923,
T or C allele in the SNP rs1151009,
G or A allele in the SNP rs2125173, and
G or C allele in the SNP rs161959.

6. The kit as claimed in claim 5, wherein the myopia comprises a spherical refraction ≦−6 D.

7. The kit as claimed in claim 5, wherein the kit further comprises a polymerase, deoxynucleotides, an enzyme or a buffer.

8. The kit as claimed in claim 5, wherein the probe or primer is detectably labeled.

9. The kit as claimed in claim 5, wherein the sample comprises blood, an amniotic fluid, cerebrospinal fluid, tissue from skin, muscle, buccal or conjunctival mucosa, placenta, or gastrointestinal tract.

10. The kit as claimed in claim 5, wherein the subject comprises mammals.

11. The kit as claimed in claim 5, wherein the subject is a human.
